(19) United States
(12) Patent Application Publication
(10) Pub. No.: US 2010/0145169 A1
(43) Pub. Date: Jun. 10, 2010

(54) METHODS AND SYSTEMS FOR DETECTING A CONDITION OF COMPARTMENT SYNDROME

(75) Inventors: Luya Li, Coquitlam (CA); Qichao Zhu, Burnaby (CA); Phaik Koay, Vancouver (CA); Matthew Peter Walls, New Westminster (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: URODYNAMIX TECHNOLOGIES LTD., Vancouver, BC (CA)

(21) Appl. No.: 12/517,054

(22) PCT Filed: Nov. 30, 2007
(86) PCT No.: PCT/CA2007/002151

§ 371 Date: Dec. 11, 2009

Related U.S. Application Data
(60) Provisional application No. 60/868,319, filed on Dec. 1, 2006.

(51) Int. Cl. A61B 5/1455

(52) U.S. Cl. 600/323

(57) ABSTRACT

Methods and systems for detecting and alerting one to a condition of Compartment Syndrome provide for determining concentration data of biochemical compounds in tissues, preferably at a shallow depth beneath the skin; analysing the concentration data to detect a condition of Compartment Syndrome; and triggering an alarm if a condition of Compartment Syndrome is detected. The concentration of biochemical compounds may be measured using Near Infrared Spectroscopy. The biochemical compounds may comprise at least one compound from the group consisting of Hemoglobin, Oxygenated Hemoglobin, Cytochromes and Myoglobin.

### CROSS REFERENCE TO RELATED APPLICATION

This invention claims the benefit of U.S. Provisional Application Ser. No. 60/868,319 filed Dec. 1, 2006 and entitled METHODS AND SYSTEMS FOR DETECTING A CONDITION OF COMPARTMENT SYNDROME which is hereby incorporated herein by reference in its entirety.

### TECHNICAL FIELD

This invention relates to methods and systems for detecting and alerting one to a condition of Compartment Syndrome (CS) in humans or other mammals. Embodiments of the invention apply Near Infrared Spectroscopy (NIRS) to analyse biochemical compounds in tissues at a shallow depth beneath the skin. This invention has particular application to the monitoring of critically ill or post-operative patients for early detection and diagnosis of CS.

### BACKGROUND

CS is caused by elevated compartmental pressure, for example, intra-abdominal pressure (TAP), or intra-compartment pressure (ICP) in a closed fascial space, typically in a limb. Elevated compartmental pressure impairs tissue perfusion, which leads to ischemia and pain, and may result in organ failure and death if left untreated.

CS commonly results from soft tissue injury. Trauma or critically ill patients are at risk of developing CS. For example, patients who have had a laparotomy for major trauma are at risk of developing abdominal CS.

The longer that CS is left untreated, the higher the risk of organ dysfunction and death. Thus, early detection of symptoms which indicate a trend toward CS or the development of CS is crucial to maintaining normal organ function and ensuring patient survival.

One way of monitoring patients for CS is to measure the compartmental pressure; a high compartmental pressure is a potential indicator of CS. It is possible to measure pressure by inserting a pressure monitor (such as a pressure transducer or manometer) into the region of the body to be studied. Typically, IAP is monitored by measuring bladder pressure, and ICP is monitored by measuring pressure in a compartment of a limb. However, such methods of measuring pressure are invasive. Such methods also must be repeated intermittently, such as every few hours or once a day, if one wishes to monitor changes in pressure over time.

NIRS is a technique which involves emitting near infrared (NIR) light and receiving the NIR light after it has passed through a tissue or other medium of interest. NIRS can be applied to study and monitor biochemical compounds in the body. Emitted NIR light penetrates skin and other tissues and some of it is absorbed by biochemical compounds which have an absorption spectrum in the NIR region. NIR light which is not absorbed is scattered. Each biochemical compound has a different absorption spectrum. It is possible to estimate the concentration of biochemical compounds in the tissues by measuring characteristics of NIR light that has been detected after it has passed through the tissues.

As discussed in Varela, J. Esteban et al., Near-infrared spectroscopy reflects changes in mesenteric and systemic perfusion during abdominal compartment syndrome, Surgery Vol. 129, No. 3, pp. 363-370, (2001) a study was conducted on swine for which abdominal CS was induced. An NIRS probe was inserted into the stomach for measurement of gastric oxygen saturation. Another NIRS probe was placed upon the skin surface of the left front limb for measurement of muscle tissue oxygen saturation. The study found that NIRS could detect changes in gastric oxygen saturation and muscle tissue oxygen saturation in the swine, which correlated with mesenteric perfusion and systemic perfusion, respectively.

However, there exists a need for a minimally-invasive method and system for early detection and diagnosis of the onset or potential onset of CS. There also exists a need for a method and system which alerts one to the onset or potential onset of CS.

### SUMMARY

This invention provides methods and systems for detecting and alerting one to a condition of CS in a human or other mammal. A condition of CS includes one or more of the following:
a condition which may indicate a trend toward CS;
a condition which may indicate the onset of CS; and
a condition which may indicate that CS is occurring.

One aspect of the invention provides methods which detect a condition of CS by measuring the concentration of biochemical compounds in the tissues, preferably at a shallow depth beneath the skin (in some embodiments at depths of 40 mm or less), and monitoring trends in the concentration of these biochemical compounds. The biochemical compounds may comprise one or more compounds from the group consisting of deoxygenated hemoglobin (Hb), Oxygenated Hemoglobin (HbO₂), Cytochromes (Cyt), and Myoglobin (Mb). The concentration data for the biochemical compounds may be acquired by applying NIRS. Trends in the concentration data correlate to changes in compartmental pressure, which may be indicative of a condition of CS. Therefore, an analysis of the trends in the concentration data may be used to detect a condition of CS.

Another aspect of the invention provides methods which detect a condition of CS by analysing the concentration of biochemical compounds in the tissues, preferably at a shallow depth beneath the skin, to provide an estimated value of compartmental pressure. Optionally, the analysis extrapolates from an initial measurement of compartmental pressure to arrive at an estimate of compartmental pressure.

Another aspect of the invention provides methods to activate an alarm if a condition of CS has been detected. Different levels of alarms may be provided to signify different severity levels for each condition of CS.

A separate aspect of the invention provides systems to detect a condition of CS, comprising a data monitoring subsystem which processes and analyses concentration data of biochemical compounds in the tissues. The concentration data may be obtained through a data acquisition subsystem, such as a NIRS subsystem. The biochemical compounds which are monitored may include at least one compound from the group consisting of Hb, HbO₂, Cyt and Mb. The data monitoring subsystem stores concentration data at periodic intervals. The data monitoring subsystem analyses the concentration data by performing one or more of the following:
monitoring trends in the concentration data; and
determining an estimate of compartmental pressure based on the concentration data.

Another aspect of the invention provides a system comprising an alarm which is triggered if an analysis of the trends in the concentration of biochemical compounds or the estimate of compartmental pressure indicates a condition of CS.

Another aspect of the invention provides media containing instructions, which, when executed by a data processor, cause the data processor to analyse the concentration data of biochemical compounds, and to trigger an alarm if the analysis indicates a condition of CS.

Further aspects of the invention and features of specific embodiments of the invention are described below.

### BRIEF DESCRIPTION OF THE DRAWINGS

In drawings which illustrate non-limiting embodiments of the invention,

FIG. 1 is a flow chart illustrating a method for detecting and alerting one to a condition of CS;

FIG. 2 is a flow chart illustrating a specific implementation of the method of FIG. 2;

FIG. 3 is a block diagram illustrating a system for detecting and alerting one to a condition of CS;

FIG. 4 is a block diagram illustrating a specific implementation of the system of FIG. 3; and,

FIG. 5 is a block diagram illustrating a data monitoring subsystem which may be used in the system of FIG. 4.

### DESCRIPTION

Throughout the following description, specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

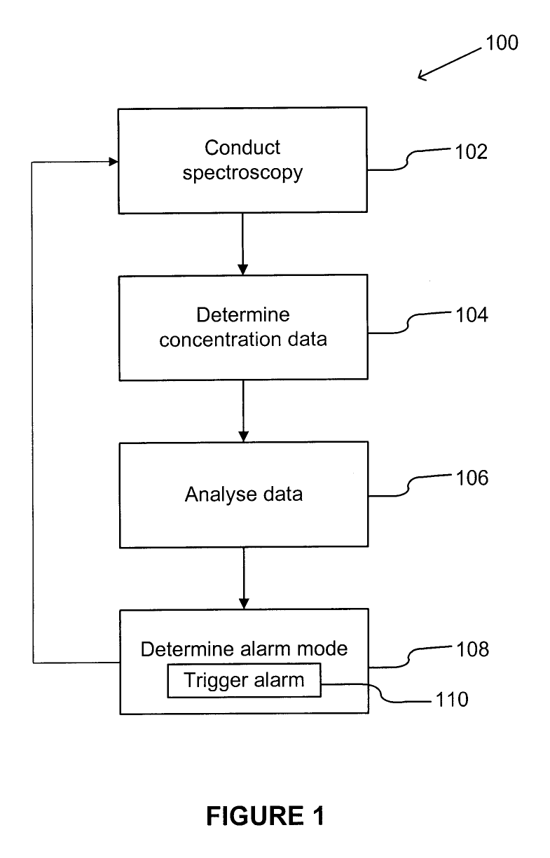

FIG. 1 illustrates a method 100 for detecting and alerting one to a condition of CS. (“A condition of CS” is defined in the Summary of the disclosure, above.) At block 102, spectroscopy, preferably absorption spectroscopy, is conducted on a patient to detect and measure biochemical compounds in the tissues. Absorption spectroscopy is the technique of emitting electromagnetic radiation to study matter which preferentially absorbs the radiation at a given spectrum. NIRS is a form of absorption spectroscopy which may be used for detecting biochemical compounds which have an absorption spectrum in the NIR region.

In one embodiment of the invention, NIRS may be conducted on a patient by directing NIR light at the skin of the region to be monitored for CS (e.g. abdomen, lower leg, or brain), and detecting and measuring the NIR light that is scattered back through the skin. Preferably, the NIR light provided for conducting NIRS is targeted at measuring biochemical compounds in the tissues at a shallow depth beneath the skin. As will be discussed in further detail below, this may be achieved by placing an NIR transmitter and an NIR receiver close together on the surface of the skin, so as to detect NIR back scattered light from a shallow depth in the tissues.

Method 100 proceeds to block 104, where the scattered light that is detected at block 102 is analysed to obtain concentration data for biochemical compounds in the tissues. The compounds may comprise at least one compound from the group consisting of Hb, HbO₂, Cyt, and Mb.

The concentration data is analysed at block 106. The analysis may comprise monitoring trends in the data, for example, monitoring a change in concentration value relative to an initial concentration value, or monitoring the first derivative of the concentration with respect to time. Such trends generally correlate to changes in compartmental pressure. Therefore, based on an analysis of these trends, it may be determined if the patient has a condition of CS.

The analysis of data performed at block 106 may comprise determining an estimate for compartmental pressure, based on the concentration data. An estimate for compartmental pressure which is greater than a threshold value may indicate a condition of CS.

If a condition of CS is detected, an alarm which corresponds to the level of severity of the condition of CS is selected at block 108. It is also possible to provide only one alarm which corresponds to any or all conditions of CS. After selecting the alarm, a corresponding alarm is triggered at block 110.

The steps described above may be repeated continuously for so long as it is desired to monitor the patient for CS.

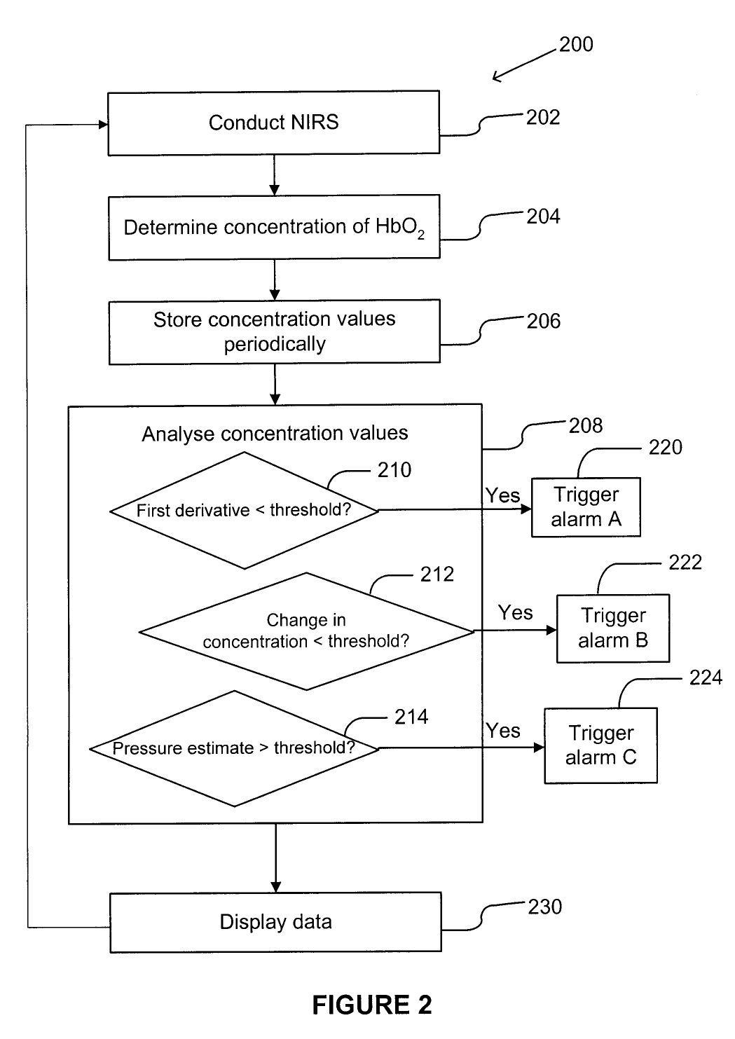

FIG. 2 illustrates a method 200 which is a specific implementation of method 100 of FIG. 1. At block 202, NIRS is conducted on a patient to detect biochemical compounds in the tissues, preferably at a shallow depth beneath the skin. At block 204, the data acquired from the NIRS step of block 202 is analysed to obtain concentration values for HbO₂. These concentration values are stored at periodic intervals, as illustrated at block 206.

The concentration values are analysed at block 208 to detect a condition of CS. For example, one or more of the following trends in the concentration values may be analysed:
at block 210, the first derivative of the concentration value with respect to time is compared with a threshold value which corresponds to a value indicating a condition of CS. If the first derivative is less than the threshold value, then a corresponding alarm is triggered at block 220.
at block 212, the difference between the concentration of HbO₂ and an initial concentration of HbO₂ is compared with a threshold value which corresponds to a value indicating a condition of CS. If the difference is less than the threshold value, then a corresponding alarm is triggered at block 222. Typically for HbO₂, the threshold value will be a negative value.

An estimate for compartmental pressure may be determined based on the concentration values. This estimate may be compared to a threshold value which corresponds to a value indicating a condition of CS, as shown at block 214. If the estimated pressure is greater than the threshold value, then a corresponding alarm is triggered at block 224. Optionally, an estimate for compartmental pressure is extrapolated from a measurement of initial compartmental pressure.

The concentration values and results of the analysis at block 208 may be displayed on a display, as shown at block 230. Also, the steps described above may be repeated continuously for so long as it is desired to monitor the patient for CS.

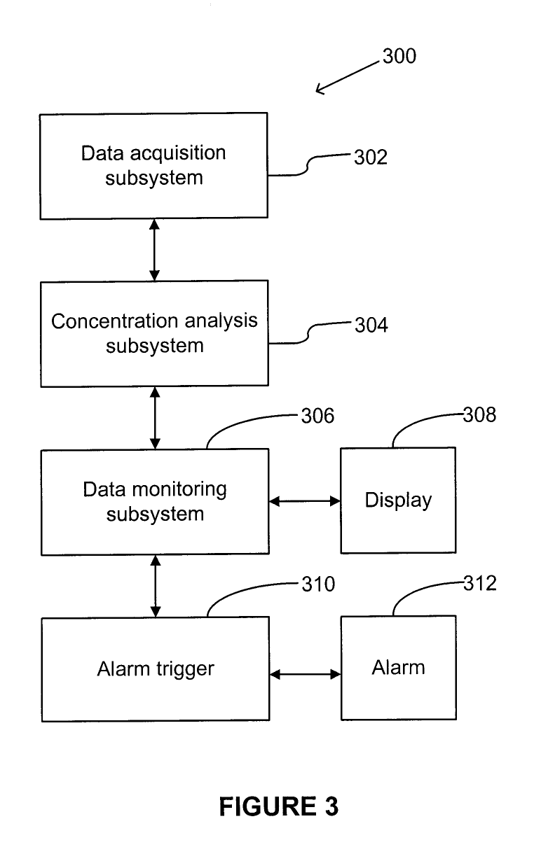

FIG. 3 illustrates a system 300 for detecting and alerting one to a condition of CS. System 300 comprises a data acquisition subsystem 302 to detect biochemical compounds in the tissues, preferably at a shallow depth beneath the skin. Data acquisition subsystem 302 may use absorption spectroscopy techniques. For example, NIRS may be conducted on a patient to detect biochemical compounds.

The data which is acquired by data acquisition subsystem 302 is analysed by a concentration analysis subsystem 304 to determine the concentration of biochemical compounds in the tissues. The compounds may comprise at least one compound from the group consisting of Hb, HbO₂, Cyt, and Mb. For example, concentration data of HbO₂ and Mb may be acquired in one embodiment of the invention.

The concentration data is input to a data monitoring subsystem 306 which stores the data at periodic intervals. Data monitoring subsystem 306 analyses the data to detect a condition of CS, by monitoring trends in the data which may be indicative of a condition of CS, and/or determining an estimate of compartmental pressure.

Data monitoring subsystem 306 is connected to a display 308 for displaying the data. Data monitoring subsystem 306 is also connected to an alarm trigger 310 which activates an alarm 312 if it is determined by the data monitoring subsystem that the trends in data or the estimate of compartmental pressure indicate that the patient has a condition of CS.

Alarm 312 may comprise, for example, an audible alarm (e.g. bell or beep), visual alarm (e.g. light), or a combined visual and audible alarm. Alarm trigger 310 may be wired to alarm 312 or it may transmit a wireless message which activates alarm 312 on a wireless receiving device (e.g. Personal Digital Assistant, pager, or cellular phone). Other types of alarms are possible for providing notification of a condition of CS. Although only one alarm is illustrated, a plurality of different alarms may be provided, each signifying a different level of warning. For example, a particular alarm may be activated to indicate the early stages of CS, while another alarm may be activated if CS has progressed to a more severe stage.

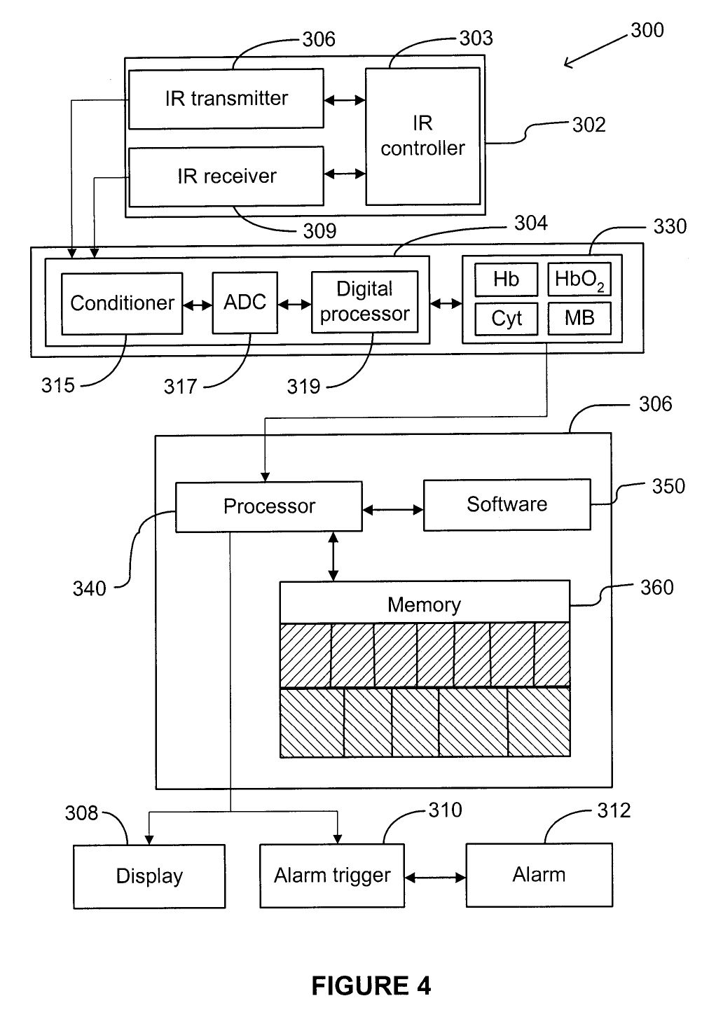

FIG. 4 illustrates a specific implementation of system 300. Data acquisition subsystem 302 is provided to conduct NIRS on a patient. Data acquisition subsystem 302 comprises an NIR transmitter 306 and an NIR receiver 309, each connected to an NIR controller 303. Although only one NIR receiver is illustrated, data acquisition subsystem 302 may comprise more than one NIR receiver 309.

Preferably, NIR transmitter 306 and NIR receiver 309 are contained in a probe or probes placed on the patient's skin. NIR transmitter 306 directs NIR light at the patient's skin. The NIR light may have one or more bands in the spectrum range of 700 to 950 nm. The transmitted NIR light penetrates the skin and other tissues and some of it is absorbed by biochemical compounds, such as proteins, which each have a different absorption spectrum in the NIR region. The NIR light which is not absorbed is scattered back through the skin, and some of this back scattered light is detected by NIR receiver 309.

It is preferable that the NIR light is targeted to detect and measure biochemical compounds in the tissues at a shallow depth beneath the skin. The depth at which biochemical compounds are detected is preferably between 10 mm to 30 mm. This depth range is approximately a function of the intensity of the NIR light source and the distance between NIR transmitter 306 and NIR receiver 309.

Therefore, if the intensity of the NIR light source is fixed at a certain intensity level, the depth at which biochemical compounds are detected may be set by selecting an appropriate separation distance d between NIR transmitter 306 and NIR receiver 309. Generally, a decreased separation distance d results in a decreased depth. In some embodiments, separation distance d is preferably less than 60 mm. Separation distance d is preferably between 40 to 60 mm for conducting NIRS in the abdominal region, and preferably between 30 to 35 mm for conducting NIRS for the leg and brain. However, separation distance d may be greater than 60 mm or less than 30 mm in some cases. The separation distance may be selected based upon factors such as the intensity of the emitted light and characteristics of the patient, such as pigmentation, body mass index (BMI) etc.

NIR controller 303 may determine the intensity of the NIR light transmitted by NIR transmitter 306, and may set the distance separating NIR transmitter 306 from NIR receiver 309.

The NIR light which is detected and received by NIR receiver 309 is output by NIR receiver 309 in the form of an analog signal. This signal is sent to concentration analysis subsystem 304. A signal conditioner 315 conditions the analog signal to prepare it for analog to digital conversion by converter 317. For example, signal conditioner 315 may amplify and/or filter the signal at the frequencies of interest.

After the conditioned analog signal is converted to a digital signal by converter 317, digital processor 319 may perform further filtering of the signal, such as to remove signals attributable to background NIR radiation.

Digital processor 319 analyses the signal to determine concentration data 330 for biochemical compounds. The compounds may comprise at least one compound from the group consisting of Hb, HbO₂, Cyt, and Mb. Each of these biochemical compounds absorbs NIR light at a different spectrum. Thus, by comparing the spectrum of the NIR light transmitted by NIR transmitter 306 with the spectrum of the NIR light received by NIR receiver 309, concentration data 330 may be determined. For example, concentration data 330 may be determined by transmitting NIR light having a set of discrete wavelengths, and monitoring the wavelengths contained in the output signal of NIR receiver 309. Concentration data 330 is then sent to data monitoring subsystem 306.

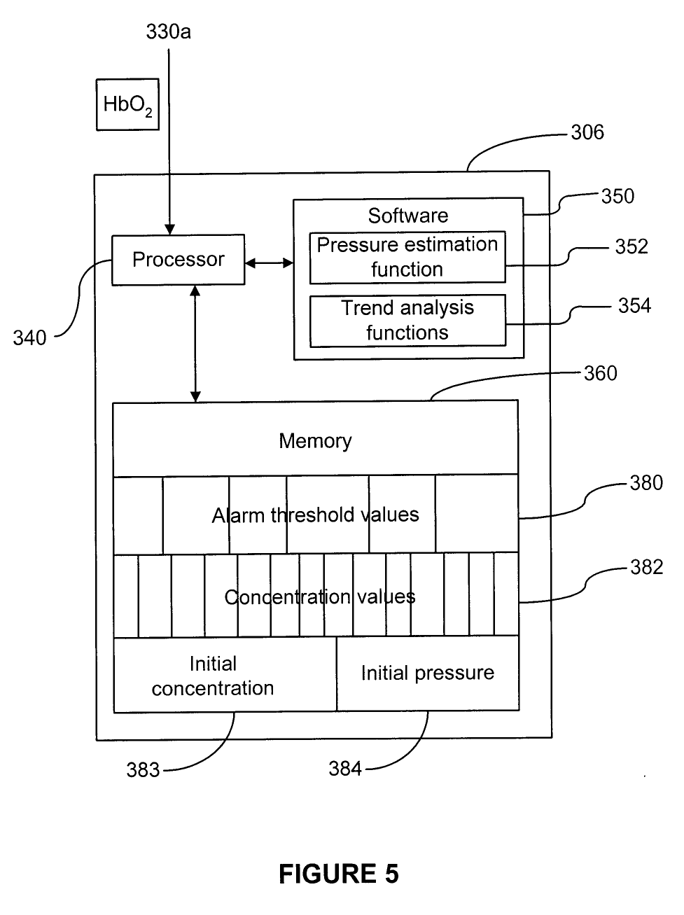

FIG. 5 illustrates, in further detail, data monitoring subsystem 306. Data monitoring subsystem 306 receives a subset of concentration data 330, namely concentration data 330a for HbO₂. Although not illustrated, data monitoring subsystem 306 may also receive concentration data 330 for one or more of Hb, Cyt and Mb. In some embodiments, alarm criteria involve concentrations or concentration trends of two or more of HbO₂, Hb, Cyt and Mb.

Data monitoring subsystem 306 comprises a processor 340, which executes instructions contained in software 350 and reads/writes data to/from memory 360. Memory 360 stores, for example, a plurality of alarm threshold values 380 corresponding to trends in the data; an initial concentration value 383 of concentration data 330a; and the last n values 382 of concentration data 330a, recorded at periodic intervals, such as every 2 minutes. If compartmental pressure is measured initially through direct means such as a pressure monitor, memory 360 may store an initial pressure value 384 of compartmental pressure.

Software 350 contains a plurality of functions related to detecting a condition of CS. This may include one or more of the following: a pressure estimation function 352 and a plurality of trend analysis functions 354. To determine whether the patient has a condition of CS, processor 340 calls and executes functions in software 350 with selected information from memory 360 as inputs to the functions.

Trend analysis functions 354 may comprise one or more of the following functions:
a function to determine and analyse the first derivative of the concentration of HbO₂ with respect to time. If the first derivative of the concentration of HbO₂ is less than a corresponding value in alarm threshold values 380, then a corresponding alarm 312 is activated by alarm trigger 310.
a function to determine if the difference between the concentration of HbO₂ and the initial concentration value 383 of HbO₂ is less than a corresponding value in alarm threshold values 380. If the difference is less than the alarm threshold, then a corresponding alarm 312 is activated by alarm trigger 310.
   Trend analysis functions may be performed for one or more other biochemical compounds instead of or in addition to HbO₂.

Pressure estimation function 352 is based on the studied correlation between compartmental pressure and the trends in concentration values of one or more biochemical compounds, for example, one or more compounds from the group consisting of Hb, HbO₂, Cyt, and Mb. Thus, an estimated value for compartmental pressure may be determined from concentration data 330. If initial pressure value 384 is measured, pressure estimation function 352 may extrapolate from initial pressure value 384 to provide an estimate of compartmental pressure at a later time.

The estimated value for compartmental pressure is compared to a corresponding value in alarm threshold values 380. If the estimated compartmental pressure is higher than the alarm threshold, a corresponding alarm 312 is activated by alarm trigger 310.

A plurality of different alarms may be provided to signify different levels of warning. For example, if the first derivative of the concentration of HbO₂ with respect to time is less than a first threshold value, a, then a corresponding alarm 312 may be activated to indicate a first level of warning. If the first derivative is less than a second threshold value, β, where β<α, then another corresponding alarm 312 may be activated to indicate a higher, second level of warning. Similarly, various corresponding alarms may be associated with different threshold values corresponding to negative changes in concentration of HbO₂.

Software 350 may comprise functions to cause alarm trigger 310 to activate corresponding alarm 312 if a condition of CS is detected. Alarm trigger 310 may be provided through a software function, such as a function contained in software 350.

Display 308 may display information related to monitoring the patient for a condition of CS, such as one or more of the following:
a plot of concentration data 330 over time;
information about the trends in concentration data 330 (e.g. first derivative with respect to time, or total change in concentration from initial concentration value 383); and
a visual indication of any of the alarms 312 that are activated by alarm trigger 310.

Although not illustrated, a device for printing out information may be provided. The device may print information displayed by display 308 or other information related to monitoring the patient for a condition of CS.

As noted above, concentration data 330 for one or more of Hb, Cyt and Mb may also be received by data monitoring subsystem 306. If such data is received, trend analysis functions 354 may comprise suitable functions to analyse trends for each of Hb, Cyt, and Mb to detect a condition of CS. For example, an increase in concentration of Mb may indicate a condition of CS. Thus, a function may compare the change in concentration of Mb from an initial value of concentration of Mb, to a threshold value. If the change in concentration is greater than the threshold value, then a corresponding alarm 310 may be activated by alarm trigger 312.

Trend analysis functions 354 may further comprise functions which analyse trends for a combination of at least two biochemical compounds from the group consisting of Hb, HbO₂, Cyt and Mb. For example, a function may consider trends in the sum of concentration values of Hb and HbO₂, which is related to the blood volume. A change in this sum which is greater than a threshold value may indicate a condition of CS.

As will be apparent to those skilled in the art in the light of the foregoing disclosure, many alterations and modifications are possible in the practice of this invention without departing from the spirit or scope thereof. For example:
while the lower limbs and abdomen are the most common sites for developing CS, the methods and systems described above may be applied to detect a condition of CS in any part of the body that may develop CS, such as the upper limbs and the brain;
an apparatus may be provided which contains one or more subsystems or devices described above; for example, data monitoring subsystem 306, display 308, alarm trigger 310 and alarm 312 may be contained in one apparatus; and
values such as alarm threshold values 380 may be written explicitly into instructions in software 350, rather than being stored in memory 360.

1. Apparatus for detecting a condition of a compartment syndrome, the apparatus comprising:
a non-invasive sensor positionable on a subject's skin to detect a concentration of one or more biochemical compounds within the subject;
a trend analysis system coupled to measure a trend in the concentration of the one or more biochemical compounds; and
an alarm system connected to trigger an alarm signal in response to an output of the trend analysis system.

2. Apparatus according to claim 1 wherein the non-invasive sensor has a greatest sensitivity to detect the one or more biochemical compounds at a subcutaneous depth of 40 mm or less.

3. Apparatus according to claim 2 wherein the subcutaneous depth is in the range of 10 mm to 30 mm.

4. Apparatus according to claim 1 wherein the one or more biochemical compounds comprises a plurality of biochemical compounds and the trend analysis system determines trends in the concentrations of each of the plurality of biochemical compounds.

5. Apparatus according to claim 4 wherein the alarm system is configured to trigger the alarm signal based on the trends in concentrations of the plurality of biochemical compounds.

6. Apparatus according to claim 1 comprising a compartmental pressure estimator system for estimating inter-compartmental pressure based on the concentration of the one or more biochemical compounds.

7. Apparatus according to claim 6 wherein the alarm system is configured to trigger the alarm signal in response to the estimated inter-compartmental pressure exceeding an inter-compartmental pressure threshold.

8. Apparatus according to claim wherein the compartmental pressure estimator system is configured to determine an estimated inter-compartmental pressure based at least in part on an initial measured inter-compartmental pressure and a difference between current and initial values of the concentration of the one or more biochemical compounds.

9. Apparatus according to claim 8 comprising a memory storing the initial measured inter-compartment pressure and an initial value of the concentration of the one or more biochemical compounds measured by the non-invasive sensor.

10. Apparatus according to claim 6 wherein the alarm system is configured to trigger a first alarm signal if the estimated inter-compartmental pressure equals or exceeds a first inter-compartmental pressure threshold and to trigger a second alarm signal if the estimated inter-compartmental pressure equals or exceeds a second inter-compartmental pressure threshold.

11. Apparatus according to claim 1 wherein the non-invasive sensor comprises a near infrared spectrometry (NIRS) sensor.

12. Apparatus according to claim 11 wherein the NIRS sensor delivers near infrared (NIR) light in one or more bands in the wavelength range of 700 to 950 nm.

13. Apparatus according to claim 11 wherein the NIRS sensor comprises a NIRS light source and a NIR detector separated by a distance in the range of 30 to 60 mm.

14. Apparatus according to claim 13 wherein the distance is in the range of 40 to 60 mm.

15. Apparatus according to claim 1 wherein the non-invasive sensor is adapted to be held in place on a subject's limb.

16. Apparatus according to claim 1 wherein the non-invasive sensor is adapted to be held in place on a subject's abdomen.

17. Apparatus according to claim 1 wherein the trend analysis system comprises a differentiator configured to evaluate a first derivative of the concentration of the one or more biochemical compounds.

18. Apparatus according to claim 17 wherein the alarm system is configured to compare the first derivative to a predetermined first derivative threshold.

19. Apparatus according to claim 1 wherein the one or more biochemical compounds detected by the non-invasive sensor include one or more of hemoglobin (Hb), oxygenated hemoglobin (HbO2), a cytochrome (Cyt) and myoglobin (Mb).

20. Apparatus according to claim 19 wherein the apparatus comprises a processor configured to compute total hemoglobin concentration by summing concentrations of Hb and HbO2 measured by the non-invasive sensor and the trend analysis system monitors a trend of the total hemoglobin concentration.

21. Apparatus according to claim 20 wherein the alarm system is configured to trigger a total hemoglobin alarm if the total hemoglobin concentration exceeds a total hemoglobin threshold.

22. Apparatus according to claim 19 wherein the alarm system is configured to evaluate at least three of the following trigger conditions:
a) a first derivative of a concentration of HbO2 is less than or equal to a first threshold;
b) a first derivative of a sum of a concentration of Hb and the concentration of HbO2 is greater than or equal to a second threshold;
c) a first derivative of a concentration of Mb is greater than or equal to a third threshold;
d) a measured change in the sum of the concentration of Hb and the concentration of HbO2 is greater than or equal to a fourth threshold;
e) a measured change in the concentration of Mb is greater than or equal to a fifth threshold;
and the alarm system is configured to trigger the alarm system if any of the evaluated trigger conditions are satisfied.

23. Apparatus according to claim 22 wherein the apparatus is configured to evaluate four of the trigger conditions.

24. Apparatus according to claim 22 wherein the apparatus is configured to evaluate five of the trigger conditions.

25. Apparatus according to claim 19 wherein the trend analysis system is configured to determine a trend for a combination of at least two biochemical compounds selected from the group consisting of Hb, HbO2, Cyt and Mb.

26. Apparatus according to claim 19 wherein the alarm system is configured to trigger the alarm signal in response to a measured change in the concentration of Mb being greater than or equal to a predetermined Mb threshold.

27. Apparatus according to claim 19 wherein the alarm system is configured to trigger a first alarm signal if a first derivative of the concentration of HbO2 is less than a first threshold and to trigger a second alarm signal if the first derivative of the concentration of HbO2 is less than a second threshold that is more negative than the first threshold.

28. Apparatus according to claim 27 wherein the alarm system is configured to trigger a third alarm signal if a difference between initial values of the concentration of HbO2 is more negative than a third threshold and to trigger a fourth alarm signal if the difference between initial values of the concentration of HbO2 is more negative than a fourth threshold that is more negative than the third threshold.

29. Apparatus according to claim 19 wherein the alarm system is configured to trigger a first alarm signal if a difference between initial values of the concentration of HbO2 is more negative than a first threshold and to trigger a second alarm signal if the difference between initial values of the concentration of HbO2 is more negative than a second threshold that is more negative than the first threshold.

30. Apparatus according to claim 1 comprising a display wherein the apparatus is configured to display on the display a plot showing concentration of the one or more biochemical compounds as a function of time.

31. Apparatus according to claim 30 wherein the apparatus is configured to display on the display a total difference between initial and current values of the concentration of the one or more biochemical compounds.

32. Apparatus according to claim 30 wherein the apparatus is configured to display on the display a visual indication that the alarm system has triggered the alarm signal.

33. Apparatus according to claim 1 wherein the trend analysis system and alarm system comprise a programmed data processor.

34. Apparatus for detecting a condition of a compartment syndrome, the apparatus comprising:
a non-invasive sensor positionable on a subject's skin to detect a concentration of one or more biochemical compounds in a subcutaneous region within the subject;
an inter-compartmental pressure estimator system for estimating inter-compartmental pressure based on the concentration of the one or more biochemical compounds; and,
an alarm system connected to trigger an alarm signal in response to an output of the inter-compartmental pressure estimator system.

35. Apparatus according to claim 34 wherein the noninvasive sensor comprises a near infrared spectrometry (NIRS) sensor.

36. Apparatus according to claim 35 wherein the NIRS sensor delivers near infrared (NIR) light in one or more bands in the wavelength range of 700 to 950 nm.

37. Apparatus according to claim 35 wherein the NIRS sensor comprises a NIRS light source and a NIR detector separated by a distance in the range of 30 to 60 mm.

38. Apparatus according to claim 33 wherein the inter-compartmental pressure estimator system is configured to estimate inter-compartmental pressure by extrapolation from an initial measured inter-compartmental pressure and initial values for the concentration of the one or more biochemical compounds in the subcutaneous region within the subject.

39. A method for detecting a condition of compartment syndrome, the method comprising:
non-invasive monitoring a concentration of one or more biochemical compounds at a sub-cutaneous location within a subject; and
triggering an alarm based on at least one of:
a) a trend in the concentrations of at least one of the one or more biochemical compounds; and,
b) comparison of the value of at least one of the one or more biochemical compounds to a threshold.

40. A method according to claim 39 comprising evaluating one or more of the following trigger conditions:
a) a first derivative of a concentration of HbO2 is less than or equal to a first threshold;
b) a first derivative of a sum of a concentration of Hb and the concentration of HbO2 is greater than or equal to a second threshold;
c) a first derivative of a concentration of Mb is greater than or equal to a third threshold;
d) a measured change in the sum of the concentration of Hb and the concentration of HbO2 is greater than or equal to a fourth threshold;
e) a measured change in the concentration of Mb is greater than or equal to a fifth threshold;
and triggering the alarm if any of the evaluated trigger conditions is satisfied.
